(19) United States
(12) Patent Application Publication
(10) Pub. No.: US 2006/0034825 A1
(43) Pub. Date: Feb. 16, 2006

(54) Method and anorectal formulations for treating hemorrhoidal diseases

(76) Inventor: Dan Charron, Brossard (CA)

Correspondence Address: Goudrean Gage Dubuc, 800 Square-Victoria P O Box 242, Suite 3400 Stock Excharge Tower, Montreal, QC, H4Z1E9, CA

(21) Appl. No.: 10/650,746

(22) Filed: Aug. 29, 2003

(51) Int. Cl. A61K 33/44

(52) U.S. Cl. 424/125

(57) ABSTRACT

A method and anorectal formulation for treating a hemorrhoidal disease in a patient suffering therefrom. The method comprises the local administration of a therapeutically effective amount of activated carbon to the anorectal region of a patient. The anorectal formulation comprises a therapeutically effective amount of activated carbon as an active agent. The formulation may include one or more pharmaceutically acceptable carriers with or without one or more additional active agents. Also disclosed are methods of making the anorectal formulation and kits for treating hemorrhoidal diseases.

### FIELD OF THE INVENTION

The present invention relates to the treatment of hemorrhoidal diseases. More specifically, the present invention is concerned with the anorectal application of activated carbon for treating a patient suffering from a hemorrhoidal disease.

### BACKGROUND OF THE INVENTION

Hemorrhoidal disease is a general term for anorectal lesion. There are many types of lesions that can take place from the rectum to the anus and the neighboring region thereof, such as perianal fistula, perianal abscess, anal fissure, pruritus ani and rectal prolapse. The main complaints of patients suffering from hemorrhoids are swelling, bleeding, itching, pain, burning, soreness and discomfort.

Typical examples of hemorrhoidal diseases are perianal fistula and hemorrhoids. Perianal fistula is a syrinx formed in or around the anorectal tract. In most cases, the perianal fistula is incurable by medication, and generally, surgery is necessary for its cure.

Hemorrhoids are a varix-like dilatation of the venous plexus of the anorectal region. The venous plexus of the anorectal region is congested repeatedly due to various causes such as straining at stool, constipation, pregnancy, asthma, long-time sedentary work, drinking, etc., so that the hemorrhoidal piles are formed gradually. Hemorrhoids are roughly divided into two types: internal hemorrhoids and external hemorrhoids according to the region of occurrence of the affection. Internal hemorrhoids are formed by the plexus of the superior hemorrhoidal veins above the dentate line. External hemorrhoids are formed by the inferior hemorrhoidal veins found below the dentate line and are painful when thrombosed.

Clinical signs and symptoms of hemorrhoids are bleeding, inflammation and pain. For treatment of hemorrhoids, proper medication is applied according to the symptoms. But in the case where medication provides no satisfactory effect, surgery is performed on the patient.

Prior art medicines for hemorrhoidal diseases include a suppository, an ointment and an internal medicine. The suppository and ointment for hemorrhoidal diseases contain an analgesic/antiphlogistic agent, a hemostatic agent, an astringent, a disinfectant, etc., and contain as a main ingredient adrenocortical hormones, lithospermum root extracts, hydrocortisones, morphine hydrochlorides, scopolia rhizome/opium extracts, tannic acid, cocaines, scopolia rhizome extract/tannin, etc. The internal medicines contain paraphlebon, tribenoside, etc., as a main ingredient, and are expected to work for relieving constipation, normalizing the circulation of the blood and putting down local inflammation.

The prior art also teaches a formulation for oral administration of spherical activated carbon. Spherical activated carbons for oral administration have also been used as an oral therapeutic agent for chronic renal failure.

The public has preferred the rapid relief of topical or local therapies over the longer reacting oral treatments for a variety of diseases.

There thus remains a need for an improved treatment of hemorrhoidal diseases.

### OBJECTS OF THE INVENTION

An object of the present invention is therefore to provide an improved method of treating hemorrhoidal diseases as well as anorectal formulations for treating hemorrhoidal diseases.

### SUMMARY OF THE INVENTION

More specifically, in accordance with the present invention, there is provided a method of treating a hemorrhoidal disease in a patient suffering therefrom, the method comprising local administration of a therapeutically effective amount of activated carbon to the anorectal region of the patient

In accordance with another aspect of the present invention there is provided an anorectal formulation for treating a hemorrhoidal disease in a patient in need thereof, the formulation comprising a therapeutically effective amount of activated carbon as the active agent.

In an embodiment, the anorectal formulation further comprises an ingredient selected from the group consisting of a pharmaceutically acceptable carrier, an additional active agent and a combination thereof.

In an embodiment, the activated carbon is triturated with a diluent to form an activated carbon trituration.

In accordance with a further aspect of the present invention there is provided a method of preparing an anorectal formulation for treating a hemorrhoidal disease in a patient in need thereof, the method comprising triturating activated carbon with a diluent and providing a therapeutically effective amount of the activated carbon trituration.

In an embodiment, the method further comprises mixing the activated carbon trituration with an ingredient selected from the group consisting of a pharmaceutically acceptable carrier, an active agent and a combination thereof.

In accordance with yet another aspect of the present invention there is provided a kit for treating a hemorrhoidal disease in a patient in need thereof, the kit comprising:
a support for an anorectal formulation including a therapeutically effective amount of activated carbon as the active agent; and
an applicator for locally applying the activated carbon to the anorectal region of the patient.
   Definitions

The terms “anorectal region”, “anorectal area”, “anal region” or “anal area” should be construed herein to include the anus and the rectum, as well as all the regions of the anus, internal as well as external, such as and without limitation: the anal crypt, the anal gland, the external sphincter and the venous plexus below the dentate line, and also all the regions of the rectum such as and without limitation: the internal sphincter and venous plexus above the dentate line.

The term “pharmaceutically acceptable carrier” or “pharmaceutically acceptable vehicle” should be construed herein to include materials that are suitable for anorectal, external, intra-anal or intra-rectal or topical or local administration to the anorectal region that are not biologically or otherwise undesirable, i.e., that may be administered to an individual along with an active agent without causing any undesirable biological effects or interacting in a deleterious manner with any of the other components of the anorectal formulation in which it is contained.

The term “therapeutically effective amount” should be construed herein to include a nontoxic but sufficient amount of the agent or ingredient to provide the desired therapeutic effect. The exact amount required will vary from subject to subject, depending on the age, weight, and general condition of the subject, the severity of the condition being treated, and the like. Thus, it is not possible to specify an exact “effective amount.” However, an appropriate “effective” amount in any individual case may be determined by one of ordinary skill in the art using only routine experimentation.

The term “trituration” or “dry attenuation” should be construed herein as the grinding of substances such as plants, herbs and powders using a mortar and pestle for example or with other suitable devices. It is the primary mode of mixing the ingredients used in preparation of powdered dilutions in homeopathy.

The term “Potency” should be construed herein to refer to the concentration of the active agent after trituration with a diluent. For example, mixing 1 part of an active agent with 9 parts diluent yields a 1× Potency, which refers to 1/10th of the original concentration. Mixing 1 part of this 1× Potency with 9 parts diluent yields 2× Potency, which refers to 1/100th of the original concentration. Mixing 1 part of this 2× Potency with 9 parts diluent yields 3× Potency, which refers to 1/1000th of the original concentration. This process is repeated until the desired potency is attained.

The terms “ingredient”, “carrier” and “additional active agent” should be construed to include both the singular and the plural forms thereof.

The term “patient” should be construed herein to include humans as well as other mammals.

The term “local” should be construed herein to include topical, external application as well as internal application to the anorectal region.

The term “treatment” should be construed to also include without limitation: cleansing, disinfection and/or adsorption.

An advantage of the present invention is that it provides a relatively quick method of treating hemorrhoids that substantially minimizes any potential side effects.

Other objects, advantages and features of the present invention will become more apparent upon reading of the following non-restrictive description of embodiments thereof, given by way of example only.

### DESCRIPTION OF THE EMBODIMENT

An embodiment of the invention will be herein described so as to only exemplify the invention and not limit the scope thereof.

Activated carbon (also known as active carbon, activated charcoal, vegetable charcoal or carbo vegetabilis) is an amorphous form of carbon characterized by high adsorptivity for many gases, vapors and colloidal solids. The carbon is obtained by the destructive distillation of wood, coconut shell, nut shells, animal bones, or other carbonaceous material or the destruction of various vegetable or organic matter treated to increase its adsorptive power. It is “activated” by heating to 800-900° C. with steam or carbon dioxide, which results in a porous internal, honeycomb-like structure. For producing spherical activated carbon, there can be used suitable raw materials which are easily available, such as wood, sawdust, coal, coconut shell, coconut shell flour, petroleum or coal pitches, or organo-synthetic high polymeric substances. A process of carbonizing the raw material and activating the obtained carbon produces the spherical activated carbon. Various processes for activation such as a steam activation process, chemical activation process, an air activation process, a carbon dioxide activation process among others known to those having skill in the art can be used.

In one non-limiting embodiment, the activated carbon used as an active agent in the present invention is in a powdered state and of pharmacopoeia grade to ensure highest purity and sanitation.

A non-limiting example of a powdered activated carbon used in the present invention is the Norit® E supra USP, which is a medicinal activated carbon of 100% natural origin. This carbon has been processed to meet purification standards required for medicinal use and has a high adsorption capacity. The specification and characteristics of the Norit® E supra USP powdered activated carbon are detailed in Tables 1 and 2 respectively.

TABLE 1
Norit ® E supra USP SPECIFICATION
a) Pharmacopoeia
NORIT E SUPRA USP passes the tests required by the United
States Pharmacopeia (ed. 23).

| | | |
|---|---|---|
| Requirements: | | |
| Microbial limits | | passes |
| Reaction (pH) | | neutral |
| Loss on drying | max. | 15.0 |
| Residue on ignition | max. | 4.0 |
| Acid soluble substances | max. | 3.5 |
| Chloride | max. | 0.2 |
| Sulphate | max. | 0.2 |
| Sulphide | | passes |
| Cyanogen compounds | | passes |
| Heavy metals | max. | 0.005 |
| Uncarbonized constituents | | passes |
| Adsorptive power: | | |
| Alkaloids | | passes |
| Dyes | | passes |

b) NORIT

| | | |
|---|---|---|
| Moisture (as packed) | max. | 10 w/w |
| Acid soluble matter | max. | 1.0 w/w |
| Methylene blue adsorption | min. | 14 g/100 g |

TABLE 2
Norit ® E supra USP Characterestics:

| | | |
|---|---|---|
| Moisture(as packed) | 8 | w/w |
| Methylene blue adsorption | 18 | g/100 g |
| Acid soluble matter | 0.5 | w/w |
| Calcium (acid extr.) | 200 | mg/kg |
| Iron (acid extr.) | 200 | mg/kg |
| Magnesium (acid extr.) | 100 | mg/kg |
| Chloride (acid extr.) | 0.2 | w/w |
| Ash content | 4 | w/w |
| pH | 7 | |
| Particle size > 150 gm | 3 | w/w |
| Apparent density (tamped) | 350 | kg/m³ |
| Internal surface area (B.E.T.) | 90 | m²/g |
| | 0 | |

Of course other Norit® activated carbon products may be used within the scope of the invention as well as any other type or brand of activated carbon.

Organic activated carbon may also be used within the scope of the present invention.

Advantageously, the activated carbon used in the present invention is compliant with monograph 24 of the current edition of the USP.

In the present method of treating hemorrhoidal diseases, activated carbon is locally or topically administered to the anorectal region of a patient suffering from hemorrhoidal diseases.

The particular area of the anorectal region on which the activated carbon will be applied to will vary in accordance with the particular type of hemorrhoidal disease. Hence, local administration to the anorectal region includes external administration, intra-anal administration such as to the venous plexus below the dentate line and/or intra-rectal administration such as to the venous plexus above the dentate line. Of course, the activated carbon of the present invention can be applied to other areas of the anorectal region as is deemed necessary for a particular treatment.

In accordance with one aspect of the present invention, there is provided an anorectal formulation of activated carbon to be applied to the anorectal region of a patient suffering from hemorrhoidal diseases.

A therapeutically effective amount of activated carbon is applied to the anorectal region alone or in a mixture with another ingredient such as one or more pharmaceutically acceptable carriers and/or one or more additional active agents.

In one embodiment, the method of preparing the anorectal formulations of the present invention consists of triturating powdered activated carbon with a suitable diluent to form an activated carbon trituration. In an embodiment the diluent used is lactose (such as Lactose USP for example) and/or sucrose.

Combining the activated carbon or a trituration thereof with one or more carriers and/or with one or more other active agents requires mixing the activated carbon or trituration thereof with the aforementioned ingredients.

Specifically, the activated carbon or tituration thereof is mixed with the other ingredients until the activated carbon is completely homogeneous in the mixture and it is no longer visible or removable in its powdered state (without unreasonable complications). Usually, the mixture is completely black or dark gray or army gray in appearance and relatively viscous. When mixing, the mixture's color is grayish as air pouches form. With time the air leaves the mixture and its color darkens.

In one embodiment, when the activated carbon trituration is of potency 1× according to Class F of the HPUS it is found in the mixture anywhere from about 1% to about 99% weight in weight (w/w) with the other ingredient or ingredients forming the remaining 1 to 99% of the mixture.

In another embodiment, when the activated carbon trituration is of a potency of 2×, it is found in the mixture anywhere from about 10% to about 99% weight in weight (w/w) with the other ingredient or ingredients forming the remaining 1% to 90% (w/w) of the mixture.

A variety of anorectal formulations for local or topical application, to the external area of the anorectal region as well as the intra-anal and/or intra-rectal regions, comprising activated carbon are provided by the present invention. These anorectal formulations can include activated carbon alone or in a combination or mixture with another ingredient such as one or more pharmaceutically acceptable carriers and/or one or more active agents.

Advantageously, the entire mixture or combination of the present invention has a melting point that is such that it will not leak out of the area to which it is applied.

The anorectal formulation of the present invention can be provided in a variety of forms as can be appreciated by the skilled artisan. For example, the anorectal formulations are in the form of a preparation, a suppository, a pad or a combination of the foregoing.

In the case of a preparation, the anorectal formulation is provided in the form of a cream, an ointment, a lotion, a balm, a salve, an emollient, a liniment, an unguent, a demulcent, a cerate, a foam, a liquid, a viscous liquid, a powder or a combination of the foregoing and the like as will be understood by the skilled artisan.

A preparation is made from mixing the activated carbon with one or more carriers and with or without additional active agents. Mixing is complete when the activated carbon is completely integrated into the preparation, which is usually of a completely black or dark gray or gray uniform color.

A homeopathic preparation is made by triturating activated carbon with Lactose USP, according to the decimal or centesimal scale described in the Homeopathic Pharmacopeia of the United States (Class F Triturations). This activated carbon trituration is then mixed with one or more carriers and with or without additional active agents. Mixing is complete when activated carbon trituration is completely integrated into the preparation, which is usually of a completely black or dark gray or gray uniform color.

A suppository is made by methods known to those having skill in the art and includes activated carbon alone or the combination of activated carbon with one or more carriers and with or without additional active agents.

The pads of the invention include activated carbon alone or in a combination of activated carbon with one or more carriers and with or without additional active agents.

The homeopathic pads and suppositories of the invention include triturating activated carbon with Lactose USP, according to the decimal or centesimal scale described in the Homeopathic Pharmacopoeia of the United States (Class F Triturations). The activated carbon trituration is then mixed with one or more carriers and with or without additional active ingredients.

In another embodiment, activated carbon is applied directly without a carrier and with or without additional active agents.

Advantageously, the carriers of the present invention are viscous thus substantially avoiding the anorectal formulation from leaving the anorectal region.

As will be understood by the skilled artisan, a variety of pharmaceutically acceptable carriers can be used in the context of the present invention.

Non limiting examples of pharmaceutically acceptable carriers or vehicles that are used in accordance with the present invention are: Acetone Sodium Bisulfite; Alcohol, Almond Oil, aloe barbadensis extract, aloe barbadensis gel, Anhydrous Lanolin; Ascorbyl Palmitate, Avocado Oil, Beeswax, Benzethonium Chloride, Benzoic Acid; Benzophenone-4; benzyl alcohol, Benzyl Benzoate, BHA, BHT, Calcium Phosphate Dibasic, canola oil, capryl/capramidopropyl betaine, Carboxymethylcellulose Sodium, Castor Oil, Cetyl Alcohol, Citric Acid, Cocoa Butter, Corn Oil Cottonseed oil, Diazolidinyl Urea, Edetate Disodium; Glycerin, Glyceryl Monooleate, Glyceryl Monostearate, Glyceryl Monostearate; Glyceryl Oleate, Glyceryl Stearate, grain alcohol, hydrogenated vegetable oil, Hydroxyethylcellulose; Kaolin, Lactose, Lanolin, Lanolin Alcohol, Light Mineral Oil, Magnesium Stearate, Methylparaben, Microcrystalline Wax, Mineral Oil, Olive, olive oil, palm fruit oil, palm, palm oil, Paraffin, PEG 12 Dilaurate, Peruvian Balsam, petrolatum, petroleum, Polyethylene Glycol 300, Polyethylene Wax, Polysorbate 80, Propyl Gallate; Propylene Glycol, Propylparaben, Purified Water; Shark Liver Oil; Simethicone; Sodium Benzoate, Sodium Citrate, Sodium Lauryl Sulfate, Sorbitan Sesquioleate, Soybean oil, Sucrose, starch, Stearyl Alcohol Sweet Almond Oil, Thyme Oil, Tocopherol; tocopheryl acetate, Trisodium EDTA, Vitamin E, Water, wax, White Petrolatum, White Wax, and Xanthan Gum or combinations of the foregoing.

Additional active agents that are used in the present invention are agents that either treat hemorrhoidal disease on their own or have a variety of synergistic or enhancing effects on the activated carbon. The additional active agents of the present invention may be used in variable potencies or dilutions, as will be understood by those having skill in the art.

Non limiting examples of additional active agents are Acidum Muriaticum (Hydrochloric Acid), Aesculus hippocastanum (Horse Chesnut), Alcloxa, Aluminum hydroxide gel, Arctium lappa, Benzocaine, Benzyl, Bryonia alba (White Bryony), Calamine, Calcarea Fluorica (Calcium Fluoride), Calcarea Phosphorica (Phosphate of Calcium), Calendula officinalis, Camphor, Cocoa butter, Cod liver oil, Collinsonia canadensis (Stone-Root), Dibucaine hydrochloride, Dibucaine, Dyclonine hydrochloride, Echinacea, Ephedrine sulfate, Epinephrine hydrochloride, Epinephrine, Ferrum Phosphoricum (Phosphate of Iron), Glycerin, Graphites (Black Lead), Hamamelis virginiana, Hard fat, Hydrastis Canadensis, Hypericum, Juniper tar, Kali carbonicum (Potassium Carbonate), Kali Phosphoricum (Phosphate of Potassium), Kaolin, Lanolin, Lidocaine, Lycopodium clavatum (Clubmoss), Lycopodium, Magnesia Phosphorica (Phosphate of Magnesium), Menthol, Millefolium (Yellow Dock), Mineral oil, Natrum Phosphoricum (Phosphate of Soda), Nitricum acidum (Nitric Acid), Nux vomica (Quaker Button), Paeonia officinalis (Peony), Paeonia officinalis, Petrolatum, Phenylephrine hydrochloride, Plantago, Pramoxine hydrochloride, Quercus, Ratanhia, Resorcinol, Resorcinum, Shark liver oil, Solidago, Sulphur (Sublimed Sulphur), Tetracaine hydrochloride, Tetracaine, Topical starch, Urtica urens, Viburnum Opulus, White petrolatum, Witch hazel, Zinc oxide and any combination thereof.

Advantageously, the anorectal formulations of the present invention should be applied to the anorectal region and left in place for enough time to allow adsorption. In an embodiment, the activate carbon formulation is left in place for at least two (2) hours.

As described above, the present invention also provides various methods of preparing an anorectal formulation for treating a hemorrhoidal disease in a patient in need thereof.

In one embodiment, the activated carbon is mixed as explained above with one or more ingredients. As will be understood by the skilled artisan, a therapeutically effective amount of activated carbon is provided for each formulation and dosage thereof for application.

In another embodiment, the activated carbon is triturated with a diluent such as lactose or sucrose. The activated carbon trituration is then mixed with another ingredient as described herein. Again, a therapeutically effective amount of activated carbon is provided.

In one embodiment, the anorectal formulations of the invention are delivered to the anorectal region with a finger.

More practically, the anorectal formulations disclosed herein are applied with a tube applicator, preferably broached and with opening at the end. This applicator is commonly referred to as a “pile pipe”. The cream or ointment may also be delivered with a large syringe and injected inside the rectum.

Therefore, the present invention also provides a variety of kits for treating a hemorrhoidal disease in a patient in need thereof.

The kits of the present invention comprise a support for the activated carbon or the formulations thereof as disclosed herein and an applicator. The applicator is used to locally apply a therapeutically effective amount of the activated carbon alone or in combination with one or more ingredients to the anorectal region of the patient.

As will be apparent to the person having skill in the art, a wide variety of supports to carry the activated carbon or anorectal formulation can be used within the context of the present invention. Non-limiting examples of such supports are a container, a capsule, a bottle, a jar, a can, a canister, a package, a packet, and a tube.

Furthermore, a wide variety of applicators can be contemplated by those having skill in the art within the context of the present invention. Non-limiting examples of such applicators are a pipe, a tube, a syringe, a conduit, a hose, a swab, and a longitudinal insert member.

The kits of the present invention may further comprise a delivery device for delivering the activated carbon or the formulation thereof from the support to the anorectal region when the applicator is at the anorectal region of the patient. Therefore, the kits of the invention may include a variety of combinations of supports, delivery devices and applicators.

The skilled artisan will easily appreciate that a wide variety of delivery devices can be used to deliver the activated carbon from the support to the anorectal region via the applicator. Non-limiting examples of the such delivery devices include a pump and a spray. The foregoing are convenient when the formulation is provided in liquid form.

### EXAMPLES

The invention will now be further described by way of non-limiting examples whose purpose is to only exemplify specific embodiments of the invention and not to limit the scope thereof.

### Example 1

### Effect on Hemorrhoidal Diseases with Pain, Burning and Discomfort

A 0.6 oz (17 g) application of cream containing activated carbon was administered with a tube applicator to a patient (male, 44 years of age) suffering from chronic hemorrhoids and exhibiting symptoms of pain, burning and general discomfort in the anorectal area. The cream was left in place while patient was sleeping and no cleaning or defecation took place during that time period (approximately 10 hours). The next morning the cream had been completely absorbed into the skin and/or was incorporated into the stool. A prominent improvement of the condition was noted immediately, the pain and burning were almost completely eliminated. During the course of the day following the treatment, what remained of the symptoms also disappeared. Such a rapid and dramatic improvement of the patient's hemorrhoidal situation had not come about with any other treatment available on the market.

### Example 2

### Effect on Hemorrhoidal Diseases with Pain, Itch and Soreness

A 0.3 oz (8.57 g) application of ointment containing activated carbon was administered with the finger to a patient (female, 51 years of age) suffering from hemorrhoidal and other anorectal diseases, comprising pain, itching, and soreness in the anorectal area. The cream was left in place undisturbed for 6 hours during the day (time during which the patient avoided cleaning and defecation). The patient's following bowel movement showed her stool as of a black color and her symptoms had dramatically improved, she showed continuous relief from the pain, itching and soreness felt previously. The situation continued to improve gradually. The next day, very little of the symptoms remained and the hemorrhoids had visibly shrunk. 48 hours following the treatment, no more symptoms were felt by the patient and the hemorrhoids had been visibly reduced to less than one fifth their original size (i.e. size before the treatment had begun). The treatment proved to be far more effective than conventional treatment.

It is to be understood that the invention is not limited in its application to the details of construction and parts described hereinabove. The invention is capable of other embodiments and of being practiced in various ways. It is also to be understood that the phraseology or terminology used herein is for the purpose of description and not limitation. Hence, although the present invention has been described hereinabove by way of embodiments thereof, it can be modified, without departing from the spirit, scope and nature of the subject invention as defined in the appended claims.

What is claimed is:

1. A method of treating a hemorrhoidal disease in a patient suffering therefrom, said method comprising local administration of a therapeutically effective amount of activated carbon to the anorectal region of said patient.

2. A method according to claim 1, wherein said activated carbon is applied to the anorectal region of said patient for a period that allows adsorption by said activated carbon.

3. A method according to claim 2, wherein said activated carbon is applied to the anorectal region of said patient for a period of at least 2 hours.

4. A method according to claim 1, wherein said local administration includes intra-rectal administration.

5. A method according to claim 4, wherein said intra-rectal administration includes administration to the venous plexus of the anorectal region.

6. A method according to claim 5, wherein said administration to the venous plexus includes administration above the dentate line of the anorectal region.

7. A method according to claim 1, wherein said local administration includes intra-anal administration.

8. A method according to claim 7, wherein said intra-anal administration is below the dentate line of the anorectal region.

9. A method according to claim 1, wherein said activated carbon is triturated with a diluent to form an activated carbon trituration.

10. A method according to claim 9, wherein said diluent is selected from the group consisting of: lactose and sucrose.

11. A method according to claim 1, wherein said activated carbon is in powdered form.

12. A method according to claim 11, wherein said activated carbon powder is of pharmacopoeia grade.

13. A method according to claim 1, wherein said activated carbon is administered in a mixture together with an ingredient selected from the group consisting of a pharmaceutically acceptable carrier, an additional active agent and a combination thereof.

14. A method according to claim 13, wherein said activated carbon is present in said mixture from about 1% to 99% w/w of said mixture and said ingredient is present from about 1% to about 99% w/w of said mixture.

15. A method according to claim 14, wherein said activated carbon is triturated with a diluent to form an activated carbon trituration, said activated carbon trituration having a potency of 1×.

16. A method according to claim 15, wherein said diluent is selected from the group consisting of: lactose and sucrose.

17. A method according to claim 13, wherein said activated carbon is present from about 10% to 99% w/w of said mixture and said ingredient is present from about 1% to about 90% w/w of said mixture.

18. A method according to claim 17, wherein said activated carbon is triturated with a diluent to form an activated carbon trituration, said activated carbon trituration having a potency of 2×.

19. A method according to claim 18, wherein said diluent is selected from the group consisting of: lactose and sucrose.

20. A method according to claim 13, wherein said carrier is selected from the group consisting of: Acetone Sodium Bisulfite; Alcohol, Almond Oil, aloe barbadensis extract, aloe barbadensis gel, Anhydrous Lanolin; Ascorbyl Palmitate, Avocado Oil, Beeswax, Benzethonium Chloride, Benzoic Acid; Benzophenone-4; benzyl alcohol, Benzyl Benzoate, BHA, BHT, Calcium Phosphate Dibasic, canola oil, capryl/capramidopropyl betaine, Carboxymethylcellulose Sodium, Castor Oil, Cetyl Alcohol, Citric Acid, Cocoa Butter, Corn Oil, Cottonseed oil, Diazolidinyl Urea, Edetate Disodium; Glycerin, Glyceryl Monooleate, Glyceryl Monostearate, Glyceryl Monostearate; Glyceryl Oleate, Glyceryl Stearate, grain alcohol, hydrogenated vegetable oil, Hydroxyethylcellulose; Kaolin, Lactose, Lanolin, Lanolin Alcohol, Light Mineral Oil, Magnesium Stearate, Methylparaben, Microcrystalline Wax, Mineral Oil, Olive, olive oil, palm fruit oil, palm, palm oil, Paraffin, PEG 12 Dilaurate, Peruvian Balsam, petrolatum, petroleum, Polyethylene Glycol 300, Polyethylene Wax, Polysorbate 80, Propyl Gallate; Propylene Glycol, Propylparaben, Purified Water; Shark Liver Oil; Simethicone; Sodium Benzoate, Sodium Citrate, Sodium Lauryl Sulfate, Sorbitan Sesquioleate, Soybean oil, Sucrose, starch, Stearyl Alcohol, Sweet Almond Oil, Thyme Oil, Tocopherol; tocopheryl acetate, Trisodium EDTA, Vitamin E, Water, wax, White Petrolatum, White Wax, Xanthan Gum and a combination thereof.

21. A method according to claim 13, wherein said additional active agent is selected from the group consisting of Acidum Muriaticum (Hydrochloric Acid), Aesculus hippocastanum (Horse Chesnut), Alcloxa, Aluminum hydroxide gel, Arctium lappa, Benzocaine, Benzyl, Bryonia alba (White Bryony), Calamine, Calcarea Fluorica (Calcium Fluoride), Calcarea Phosphorica (Phosphate of Calcium), Calendula officinalis, Camphor, Cocoa butter, Cod liver oil, Collinsonia canadensis (Stone-Root), Dibucaine hydrochloride, Dibucaine, Dyclonine hydrochloride, Echinacea, Ephedrine sulfate, Epinephrine hydrochloride, Epinephrine, Ferrum Phosphoricum (Phosphate of Iron), Glycerin, Graphites (Black Lead), Hamamelis virginiana, Hard fat, Hydrastis Canadensis, Hypericum, Juniper tar, Kali carbonicum (Potassium Carbonate), Kali Phosphoricum (Phosphate of Potassium), Kaolin, Lanolin, Lidocaine, Lycopodium clavatum (Clubmoss), Lycopodium, Magnesia Phosphorica (Phosphate of Magnesium), Menthol, Millefolium (Yellow Dock), Mineral oil, Natrum Phosphoricum (Phosphate of Soda), Nitricum acidum (Nitric Acid), Nux vomica (Quaker Button), Paeonia officinalis (Peony), Paeonia officinalis, Petrolatum, Phenylephrine hydrochloride, Plantago, Pramoxine hydrochloride, Quercus, Ratanhia, Resorcinol, Resorcinum, Shark liver oil, Solidago, Sulphur (Sublimed Sulphur), Tetracaine hydrochloride, Tetracaine, Topical starch, Urtica urens, Viburnum Opulus, White petrolatum, Witch hazel, Zinc oxide and a combination thereof.

22. An anorectal formulation for treating a hemorrhoidal disease in a patient in need thereof, said formulation comprising a therapeutically effective amount of activated carbon as the active agent.

23. An anorectal formulation according to claim 22, wherein said activated carbon is triturated with a diluent to form an activated carbon trituration.

24. An anorectal formulation according to claim 23, wherein said diluent is selected from the group consisting of: lactose and sucrose.

25. An anorectal formulation according to claim 22, wherein said activated carbon is in powdered form.

26. An anorectal formulation according to claim 25, wherein said activated carbon powder is of pharmacopoeia grade.

27. An anorectal formulation according to claim 22 further comprising an ingredient selected from the group consisting of a pharmaceutically acceptable carrier, an additional active agent and a combination thereof.

28. An anorectal formulation according to claim 27, wherein said activated carbon is present from about 1% to 99% w/w of said formulation and said ingredient is present from about 1% to about 99% w/w of said formulation.

29. An anorectal formulation according to claim 28, wherein said activated carbon is triturated with a diluent to form an activated carbon trituration, said activated carbon trituration having a potency of 1×.

30. An anorectal formulation according to claim 29, wherein said diluent is selected from the group consisting of: lactose and sucrose.

31. An anorectal formulation according to claim 27, wherein said activated carbon is present from about 10% to 99% w/w of said formulation and said ingredient is present from about 1% to about 90% w/w of said formulation.

32. An anorectal formulation according to claim 31, wherein said activated carbon is triturated with a diluent to form an activated carbon trituration, said activated carbon trituration having a potency of 2×.

33. An anorectal formulation according to claim 32, wherein said diluent is selected from the group consisting of: lactose and sucrose.

34. An anorectal formulation according to claim 27, wherein said carrier is selected from the group consisting of: Acetone Sodium Bisulfite; Alcohol, Almond Oil, aloe barbadensis extract, aloe barbadensis gel, Anhydrous Lanolin; Ascorbyl Palmitate, Avocado Oil, Beeswax, Benzethonium Chloride, Benzoic Acid; Benzophenone-4; benzyl alcohol, Benzyl Benzoate, BHA, BHT, Calcium Phosphate Dibasic, canola oil, capryi/capramidopropyl betaine, Carboxymethylcellulose Sodium Castor Oil, Cetyl Alcohol, Citric Acid, Cocoa Butter, Corn Oil Cottonseed oil, Diazolidinyl Urea, Edetate Disodium; Glycerin, Glyceryl Monooleate, Glyceryl Monostearate, Glyceryl Monostearate; Glyceryl Oleate, Glyceryl Stearate, grain alcohol, hydrogenated vegetable oil, Hydroxyethylcellulose; Kaolin, Lactose, Lanolin, Lanolin Alcohol, Light Mineral Oil, Magnesium Stearate, Methylparaben, Microcrystalline Wax, Mineral Oil, Olive, olive oil, palm fruit oil, palm, palm oil, Paraffin, PEG 12 Dilaurate Peruvian Balsam, petrolatum, petroleum, Polyethylene Glycol 300, Polyethylene Wax, Polysorbate 80, Propyl Gallate; Propylene Glycol, Propylparaben, Purified Water; Shark Liver Oil; Simethicone; Sodium Benzoate, Sodium Citrate, Sodium Lauryl Sulfate, Sorbitan Sesquioleate, Soybean oil, Sucrose, starch, Stearyl Alcohol Sweet Almond Oil, Thyme Oil, Tocopherol; tocopheryl acetate, Trisodium EDTA, Vitamin E, Water, wax, White Petrolatum, White Wax, Xanthan Gum and a combination thereof.

35. An anorectal formulation according to claim 27, wherein said additional active agent is selected from the group consisting of: Acidum Muriaticum (Hydrochloric Acid), Aesculus hippocastanum (Horse Chesnut), Alcloxa, Aluminum hydroxide gel, Arctium lappa, Benzocaine, Benzyl, Bryonia alba (White Bryony), Calamine, Calcarea Fluorica (Calcium Fluoride), Calcarea Phosphorica (Phosphate of Calcium), Calendula officinalis, Camphor, Cocoa butter, Cod liver oil, Collinsonia canadensis (Stone-Root), Dibucaine hydrochloride, Dibucaine, Dyclonine hydrochloride, Echinacea, Ephedrine sulfate, Epinephrine hydrochloride, Epinephrine, Ferrum Phosphoricum (Phosphate of Iron), Glycerin, Graphites (Black Lead), Hamamelis virginiana, Hard fat, Hydrastis Canadensis, Hypericum, Juniper tar, Kali carbonicum (Potassium Carbonate), Kali Phosphoricum (Phosphate of Potassium), Kaolin, Lanolin, Lidocaine, Lycopodium clavatum (Clubmoss), Lycopodium, Magnesia Phosphorica (Phosphate of Magnesium), Menthol, Millefolium (Yellow Dock), Mineral oil, Natrum Phosphoricum (Phosphate of Soda), Nitricum acidum (Nitric Acid), Nux vomica (Quaker Button), Paeonia officinalis (Peony), Paeonia officinalis, Petrolatum, Phenylephrine hydrochloride, Plantago, Pramoxine hydrochloride, Quercus, Ratanhia, Resorcinol, Resorcinum, Shark liver oil, Solidago, Sulphur (Sublimed Sulphur), Tetracaine hydrochloride, Tetracaine, Topical starch, Urtica urens, Viburnum Opulus, White petrolatum, Witch hazel, Zinc oxide and a combination thereof.

36. An anorectal formulation according to claim 22 in a form selected from the group consisting of a preparation, a suppository, a pad and a combination thereof.

37. An anorectal formulation according to claim 36, wherein said preparation is selected from the group consisting of: a cream, an ointment, a lotion, a balm, a salve, an emollient, a liniment, an unguent, a demulcent, a cerate, a foam, a liquid, a viscous liquid, a powder and a combination thereof.

38. A method of preparing an anorectal formulation for treating a hemorrhoidal disease in a patient in need thereof, said method comprising providing a therapeutically effective amount of activated carbon.

39. A method according to claim 38, wherein said providing includes triturating activated carbon with a diluent

40. A method according to claim 39, wherein said diluent is selected from the group consisting of: lactose and sucrose.

41. A method according to claim 39, wherein said activated carbon trituration has a potency of 1×.

42. A method according to claim 39, wherein said activated carbon trituration has a potency of 2×.

43. A method according to claim 38 further comprising mixing said activated carbon with an ingredient selected from the group consisting of a pharmaceutically acceptable carrier, an active agent and a combination thereof.

44. A method according to claim 43, wherein said activated carbon is present in said mixture from about 1% to 99% w/w of said mixture and said ingredient is present from about 1% to about 99% w/w of said mixture.

45. A method according to claim 43, wherein said carrier is selected from the group consisting of: Acetone Sodium Bisulfite; Alcohol, Almond Oil, aloe barbadensis extract, aloe barbadensis gel, Anhydrous Lanolin; Ascorbyl Palmitate, Avocado Oil, Beeswax, Benzethonium Chloride, Benzoic Acid; Benzophenone4; benzyl alcohol, Benzyl Benzoate, BHA, BHT, Calcium Phosphate Dibasic, canola oil, capryi/capramidopropyl betaine, Carboxymethylcellulose Sodium Castor Oil, Cetyl Alcohol, Citric Acid, Cocoa Butter, Corn Oil Cottonseed oil, Diazolidinyl Urea, Edetate Disodium; Glycerin, Glyceryl Monooleate, Glyceryl Monostearate, Glyceryl Monostearate; Glyceryl Oleate, Glyceryl Stearate, grain alcohol, hydrogenated vegetable oil, Hydroxyethylcellulose; Kaolin, Lactose, Lanolin, Lanolin Alcohol, Light Mineral Oil, Magnesium Stearate, Methylparaben, Microcrystalline Wax, Mineral Oil, Olive, olive oil, palm fruit oil, palm, palm oil, Paraffin, PEG 12 Dilaurate, Peruvian Balsam, petrolatum, petroleum, Polyethylene Glycol 300, Polyethylene Wax, Polysorbate 80, Propyl Gallate; Propylene Glycol, Propylparaben, Purified Water; Shark Liver Oil; Simethicone; Sodium Benzoate, Sodium Citrate, Sodium Lauryl Sulfate, Sorbitan Sesquioleate, Soybean oil, Sucrose, starch, Stearyl Alcohol Sweet Almond Oil, Thyme Oil, Tocopherol; tocopheryl acetate, Trisodium EDTA, Vitamin E, Water, wax, White Petrolatum, White Wax, Xanthan Gum and a combination thereof.

46. A method according to claim 43, wherein said additional active agent is selected from the group consisting of: Acidum Muriaticum (Hydrochloric Acid), Aesculus hippocastanum (Horse Chesnut), Alcloxa, Aluminum hydroxide gel, Arctium lappa, Benzocaine, Benzyl, Bryonia alba (White Bryony), Calamine, Calcarea Fluorica (Calcium Fluoride), Calcarea Phosphorica (Phosphate of Calcium), Calendula officinalis, Camphor, Cocoa butter, Cod liver oil, Collinsonia canadensis (Stone-Root), Dibucaine hydrochloride, Dibucaine, Dyclonine hydrochloride, Echinacea, Ephedrine sulfate, Epinephrine hydrochloride, Epinephrine, Ferrum Phosphoricum (Phosphate of Iron), Glycerin, Graphites (Black Lead), Hamamelis virginiana, Hard fat, Hydrastis Canadensis, Hypericum, Juniper tar, Kali carbonicum (Potassium Carbonate), Kali Phosphoricum (Phosphate of Potassium), Kaolin, Lanolin, Lidocaine, Lycopodium clavatum (Clubmoss), Lycopodium, Magnesia Phosphorica (Phosphate of Magnesium), Menthol, Millefolium (Yellow Dock), Mineral oil, Natrum Phosphoricum (Phosphate of Soda), Nitricum acidum (Nitric Acid), Nux vomica (Quaker Button), Paeonia officinalis (Peony), Paeonia officinalis, Petrolatum, Phenylephrine hydrochloride, Plantago, Pramoxine hydrochloride, Quercus, Ratanhia, Resorcinol, Resorcinum, Shark liver oil, Solidago, Sulphur (Sublimed Sulphur), Tetracaine hydrochloride, Tetracaine, Topical starch, Urtica urens, Viburnum Opulus, White petrolatum, Witch hazel, Zinc oxide a combination thereof.

47. A kit for treating a hemorrhoidal disease in a patient in need thereof, said kit comprising:
a support for an anorectal formulation including a therapeutically effective amount of activated carbon as the active agent; and
an applicator for applying said activated carbon to the anorectal region of said patient.

48. A kit according to claim 47, wherein said support is selected from the group consisting of: a container, a capsule, a bottle, a jar, a can, a canister, a package, a packet and a tube.

49. A kit according to claim 47, wherein said activated carbon applicator is selected from the group consisting of: a pipe, a tube, a syringe, a conduit, a hose, a swab, or a longitudinal insert member.

50. A kit according to claim 47, wherein said activated carbon applicator includes a delivery device for delivering said activated carbon from said support to the anorectal region when said applicator is at the anorectal region of said patient.

51. A kit according to claim 50, wherein said delivery device is selected from the group consisting of: a pump and a spray.
